(19) United States
(12) Patent Application Publication
(10) Pub. No.: US 2023/0398250 A1
(43) Pub. Date: Dec. 14, 2023

(54) Methods of Cleaning Enclosed Spaces of Contaminants

(71) Applicant: Blue Core Technologies, LLC, Yellow Springs, OH (US)

(72) Inventors: Kyle Bailey, McKinney, TX (US); Jason Bailey, McKinney, TX (US); Gene Batchelder, McKinney, TX (US); Keri Lestage, Dallas, TX (US)

(21) Appl. No.: 18/208,620

(22) Filed: Jun. 12, 2023

Related U.S. Application Data
(60) Provisional application No. 63/351,056, filed on Jun. 10, 2022.

(51) Int. Cl. A61L 2/22; A61L 2/24

(52) U.S. Cl. CPC A61L 2/22; A61L 2/24; A61L 2101/20

(57) ABSTRACT

A method and corresponding apparatus for cleaning an enclosed space by in situ generation and distribution of hypochlorous acid. The method includes selecting an apparatus comprising a container, placing the apparatus in the enclosed space, activating generation of hypochlorous acid from the container of the apparatus, distributing hypochlorous acid throughout the enclosed space as the hypochlorous acid is generated; and allowing the distributed hypochlorous acid to dwell in the enclosed space for an effective time.

### CROSS-REFERENCE TO RELATED APPLICATIONS

This application claims priority to U.S. Provisional Application No. 63/351,056, filed on Jun. 10, 2022, which is hereby incorporated herein by reference in its entirety.

### BACKGROUND

### 1. Field of the Invention

The present technology relates to apparatus and methods for air-borne dispersal of a cleaning agent into an enclosed space that includes contaminated surfaces therein to clean these surfaces, by remediating malodor-producing contaminants and biological load on surfaces and in the air. In one aspect, the technology relates to remediating enclosed spaces, such as vehicle cabins, hotel rooms, etc., that have a microbial and/or a viral load that may be hazardous to human health and that may lead to contagion.

### 2. Description of the Related Art

There is increasing concern about the spread of contagious diseases, whether these may be influenza, common colds, or a potentially lethal virus such as Ebola, or microbial or viral diseases that are not even known or identified at this time. For purposes of this description, microscopic fungi are included in the term “microbes.” Most of these microbes and viruses are spread through contact; a first person contacts some surface (for example, by shaking the hand of a contagious person or touches a contaminated surface) and acquires the contamination, becomes infected, and then passes it on to yet another person. This chain of infection is well-known. Some contaminants, whether microbial or viral, appear to be spread through air-borne means. This includes coughing and the emitting of a fine spray of contaminated and contagious sputum.

In a modern urban environment, one of the main means of transportation is in enclosed vehicles such as, but not limited to, aircraft, busses, trains, boats, cars, SUVs and trucks. Some of these are vehicles that are open to the general public to use, and some members of the public may have a communicable disease that is spread through microbes or viruses. Surfaces inside the cabin of the vehicle, where passengers are usually seated, may over time become heavily contaminated with live microbes and viral contaminants. Thus, these surfaces serve to spread the microbial or viral disease to other passengers through contact.

Even in non-public, personal or family transportation, one family member may be ill and could contaminate surfaces thereby passing a contagious illness to other family members. This is especially a risk where school-aged children “pick up” a microbial or viral infection from classmates at school and can then pass it on to parents and siblings through contaminated surfaces in a family vehicle. Some microbes or viruses may be long-lived, and immunity to these may not be readily achieved. Thus, there is a chance of recurrent illness. Merely wiping surfaces may not eliminate the microbial or viral load on surfaces because surfaces may not be smooth and totally accessible. For example, surfaces are often textured and may have joints and other features where microbial and viral loads may persist.

With regard to newly manufactured vehicles, the chances of a microbial or viral load on surfaces are low, unless the vehicle was contaminated during assembly. On the other hand, the chances that a “pre-owned” or “used” vehicle is contaminated and a source of potential infection, is relatively far higher. Aside from the potential health issues, there are often also aesthetic issues with pre-owned or used cars: they may have an odor in the cabin space from pets carried in the space or from the way in which they were (mis) used by the previous owners. This can have a negative impact on the resale value of the vehicle.

There is a need from a public health standpoint to clean surfaces within a passenger carrying cabin space of vehicles to reduce any microbial and/or viral load. Moreover, there is also not only a public health need to do this but also a business or economic need to remove any undesirable odors from the cabin space of public, used or pre-owned vehicles. Similarly, there is a need to reduce microbial and/or viral load from enclosed spaces used by multiples of persons in succession, such as hotel rooms or public facilities, for example.

### SUMMARY

This summary is intended to present a brief outline of some of the features of exemplary embodiments of the inventions; these and additional features are more particularly described in the Detailed Description, here below. The descriptions do not limit the scope on the inventions, which is set forth in the descriptions here below and appended.

An exemplary embodiment is of a method of cleaning an enclosed space by in situ generation and distribution of hypochlorous acid into the enclosed space; the method comprising:
selecting an apparatus comprising a container;
placing the apparatus in the enclosed space;
activating generation of hypochlorous acid from the container of the apparatus;
distributing hypochlorous acid throughout the enclosed space as the hypochlorous acid is generated; and
allowing the distributed hypochlorous acid to dwell in the enclosed space for an effective time.

In other aspects, the above exemplary embodiment may include any one or more of the following features:
wherein the step of activating generation of hypochlorous acid comprises the electrolyzing of a solution comprises chloride ions in the container of the apparatus to release hypochlorous acid;
wherein the step of distributing the generated hypochlorous acid throughout the enclosed space comprises agitating the electrolyzing solution in the container to create fine air-borne liquid droplets and vapor comprising hypochlorous acid such as to emit the fine air-borne liquid droplets and vapor from the container and into the enclosed space;
wherein the step of agitating the electrolyzing solution in the container to create fine air-borne liquid droplets and vapor comprises bubbling a gas or gas mixture at a controlled rate through the electrolyzing solution at a rate that causes the generated hypochlorous acid to become air-borne as fine air-borne liquid droplets and vapor;
wherein the step of agitating the electrolyzing solution in the container to create fine air-borne liquid droplets and vapor comprises using ultrasonic vibration of the solution to create the fine air-borne liquid droplets and vapor;
wherein the step of agitating the electrolyzing solution in the container to create fine air-borne liquid droplets and vapor comprises using an atomizer taking suction from the solution in the container to create and disperse fine air-borne liquid droplets and vapor in the enclosed space;
wherein the enclosed space includes air ducts to be cleaned, and the method further comprises establishing fluid communication between the enclosed space and the air ducts such that hypochlorous acid circulates into the air ducts to clean the air ducts;
wherein the step of activating generation of hypochlorous acid comprises electrolyzing a solution comprising chloride ions in a concentration that generates hypochlorous acid at a rate that permits treating an interior space in a time frame of about 12 to minutes while utilizing an enabling electrolyzing current supply;
wherein the step of agitating to emit fine air-borne liquid droplets comprises droplets of an average size greater than about 40 microns;
wherein the step of agitating to emit fine air-borne liquid droplets comprises droplets of an average size less than about 40 microns;
wherein the step of allowing the distributed hypochlorous acid to dwell comprises allowing to dwell from about 5 to about 20 minutes;
wherein the cleaning comprises neutralizing or at least partially remediating malodor in the enclosed space;
wherein, after the step of allowing to dwell for an effective time, the apparatus is automatically powered down to stop the generation of hypochlorous acid;
wherein the apparatus is communicating with applications software on an electronic device or a cloud computing system remote from the apparatus; wherein the applications software on the electronic device or the cloud computing system is accessible and controllable by an operator;
wherein the applications software on the electronic device or the cloud computing system is configured for storing stores identifying data about the enclosed space being cleaned, and storing date of cleaning, time of cleaning, and operator identifying data;
wherein the applications software on an electronic device or the cloud computing system is configured for providing alerts to an operator;
wherein the applications software on the electronic device or the cloud computing system is configured for allowing remote activating of generation of hypochlorous acid by an operator;
wherein the method further comprises sensing of a concentration of hypochlorous acid in the enclosed space; and
wherein the method further comprises stopping the generation of hypochlorous acid when the effective time is reached, and sending a notification via the applications software on an electronic device or the cloud computing system.

Exemplary embodiments also present a multi-stage cleaning process. This includes (but is not limited to) a first stage of using a liquid cleaner to clean surfaces of adhering “dirt” (undesirable organic or non-organic material that might adhere to varying degrees to surfaces in the interior space).

The foregoing Summary is not intended to be comprehensive and does not encompass all embodiments and all variations on the embodiments but should be instructive to a person of ordinary skill in the art.

### BRIEF DESCRIPTION OF THE DRAWINGS

The foregoing aspects and many of the attendant advantages, of the present technology will become more readily appreciated by reference to the following Detailed Description, when taken in conjunction with the accompanying simplified drawings of exemplary embodiments. The drawings, briefly described here below, are not to scale, are presented for ease of explanation and do not limit the scope of the inventions recited in the accompanying patent claims.

FIG. 1 is a schematic flow diagram of an exemplary embodiment illustrating some of the steps of the exemplary method of cleaning contaminated surfaces in an enclosed space.

FIG. 2A is an exemplary embodiment of an apparatus showing the generation of an air-borne cleaning agent (dashed lines) from its container, in this embodiment shown with an optional impeller that generates a vortex in the liquid solution.

FIG. 2B is an exemplary cutaway illustration of a vehicle showing the exemplary embodiment of the apparatus of FIG. 2A emitting an air-borne cleaning agent throughout the cabin. (Not shown is that the cleaning agent may be drawn into and recirculate in the air recirculation system of the vehicle)

FIG. 3A is a schematic illustration of an exemplary embodiment of an apparatus useful in the methods and systems for cleaning contaminated surfaces in an enclosed space, with the optional impeller.

FIG. 3B is an exploded view showing components of the apparatus of FIG. 3A when the optional impeller is used.

FIGS. 4A and B depict alternative views of another exemplary lid for an exemplary apparatus like that of FIGS. 3A and B.

FIG. 5 is a depiction of an exemplary embodiment of a “fogger and sprayer” combination device useful in an exemplary embodiment of the methods of the invention, along with text description of details illustrated.

FIG. 6 is an exemplary embodiment of a multi-stage cleaning process wherein a first stage is to clean surfaces of adhering “dirt” (undesirable organic or non-organic material that might adhere to varying degrees to surfaces in the interior space), with optional re-cleaning shown by a recycle loop, and finally a last stage of using either chlorine dioxide of hypochlorous acid to treat the interior space to neutralize malodor and malodor generating compounds.

### DETAILED DESCRIPTION

In the following non-limiting detailed descriptions of examples of embodiments of the inventions may refer to appended drawings and are not limited to the drawings, which are merely presented for enhancing explanations of features of the technology. In addition, the detailed descriptions may refer to particular terms of art, some of which are defined herein, as appropriate and necessary for clarity.

The term “cabin” as used in the specification and claims refers to an interior space containing contaminated surfaces that can readily be enclosed, for example by closing doors, windows and air vent system, if any, of the space such that air inside the space is neither withdrawn nor added to but may be recirculated. The air may be allowed to re-circulate in the cabin by activation of an air circulation system, for example, or use of a fan in the cabin.

The term “coating” or “coat” or “deposit” as is used in reference to the air-borne cleaning agent on surfaces, means that the cleaning agent dwells on (or is in contact with) the surface. The cleaning agent may be carried in or be carrying entrained fine water droplets that form a mist (or fog) containing cleaning agent (that might be dissolved) so that the air-borne mist/fog coats (or deposits on) and thereby cleans the surfaces, including fine surface textures, surface patterns, and tight interstitial spaces such as found, for example, in stitched seats and dash boards, etc. in vehicles. Of course, once the initially air-borne cleaning agent settles on a surface, it is no longer literally air-borne, but the term is used to distinguish it from any other cleaning agent, such as a liquid cleaning agent.

By “cleaning” in the context of the air-borne cleaning agent it is meant that the cleaning agent neutralizes deposits on surfaces that cause malodor and may decontaminate the surfaces by mechanisms including destroying odor-producing microorganisms on the surfaces. The term “neutralizes” means depriving a contaminant of its capability to produce a malodor, and in the case of some contaminants that include microorganisms, may include destroying the microorganism.

The term “cleaning” in the context of using a liquid cleaning agent in a multi-stage cleaning process means cleaning adhering contaminant load (organic or non-organic material) from surfaces in the interior space. Once this contaminant load has been removed, subsequent cleaning by an air-borne cleaning agent may be carried out, in the multi-stage process embodiment.

The terms “contamination” or “contamination load” when used in reference to surfaces within a cabin includes contaminants that cause a malodorous scent, for example, of decayed organic matter, fecal matter, and the like. It also includes microorganisms such as bacterial, fungal, or viral contamination.

The term “effective period of time” or “dwell time” as it relates to the time that a cleaning agent dwells in a cabin for cleaning of surfaces therein, means the time period needed to clean the interior space (or surfaces) to the required degree of cleanness of the contamination being treated. This may vary from about 10 to about 30 minutes; and may be from about 15 to about 20 minutes. More (or less) time may also be effective, depending upon the degree of cleaning (extent of contaminant load reduction) to be achieved and the nature of the chemical cleaning agent used. For example, some cabin spaces may have surfaces so heavily contaminated as to require more than one treatment, or to require that the air-borne cleaning agent dwell on surfaces for up to 8 hours, or overnight, to achieve a desired level of cleanliness and deodorization.

The term “significant reduction in contamination load” means that the contamination load of a particular contaminating species is reduced by at least 80% after cleaning in exemplary embodiments, or in some exemplary embodiments at least 98% after cleaning.

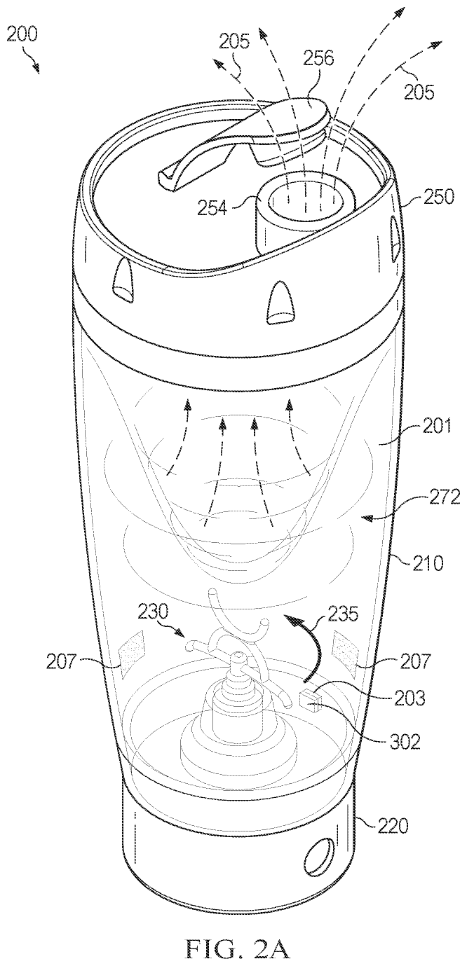

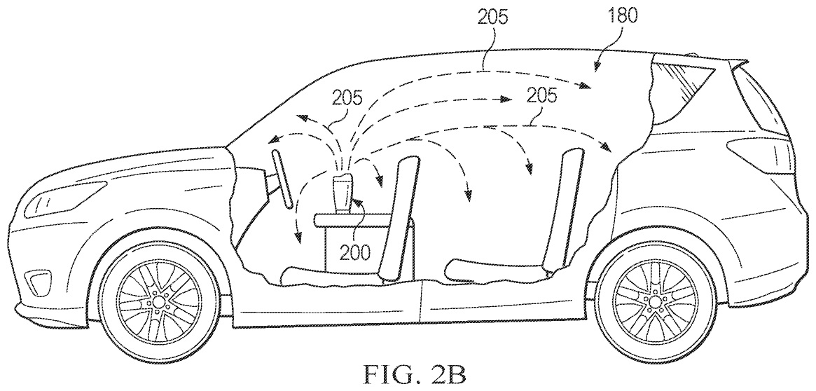

In general, the embodiments utilizing the air-borne cleaning agent have in common a method of cleaning a cabin air space and surfaces therein of a contaminant load, including malodor. This is achieved by using an apparatus that generates and releases a cleaning agent that becomes air-borne and flows into the cabin interior space. The apparatus includes a container with a lid that has an adjustably flow-restrictive opening therein. The container is configured for containing a sodium chloride solution and has a pair of electrodes configured to create hypochlorous acid (HOCl) by electrolysis of the solution. In addition, the container has means therein to cause agitation of the solution to generate a mist or fog of droplets (“microdroplets) that become air-borne and exit from the container through the opening in the lid. Due to the restricted size of the opening, pressure build up in the container forces the droplets out of the opening so that these can spread throughout the cabin interior space. This is exemplified schematically, for example in FIG. 2B, illustrating a flow of air-borne microdroplets throughout a vehicle cabin space.

In order to effectively treat a cabin and its interior surfaces within a commercially reasonable time period (for example, 10 to 20 minutes or less), the generation of HOCl must proceed at a commensurate rate. The rate and concentration of HOCl generated from the apparatus would depend on factors that include but are not limited to: sodium chloride concentration, charge supplied at the electrodes 207, the type of electrode material, the spatial distance between electrodes 207, the presence (or absence) of any electrolysis catalysts or inhibitors, and the nature of the agitation (intensity and propensity to generate microdroplets). It might be expected that microdroplets would include and be similar in composition to the solution from which they are generated. Thus, the microdroplets would include HOCl in a sodium chloride solution. The higher the HOCl concentration, within safe limits, the more rapidly the cleaning action might be expected to proceed.

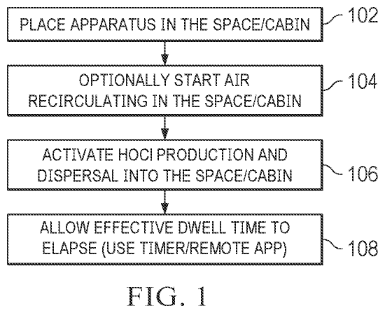

Referring to FIG. 1, an exemplary flowchart for a method of cleaning contaminated surfaces in an enclosed space, there are several straightforward steps in the system or method depicted. The steps of process 100 can be implemented in an apparatus 200, shown in more detail in FIGS. 2A, 3A, 3B and 4A and 4B that follow.

As the process 100 starts, the apparatus for generating an air-borne cleaning agent is placed in the cabin with contaminated surfaces inside to be treated (Step 102). For example, in a used car, such as exemplified in FIG. 2, all doors and windows are closed, and the air circulation system is either closed or started up if the system also requires cleaning (Step 104). Thus, air does not enter or leave the cabin except for natural flow around seals of doors and windows, which may occur in a closed cabin. When the apparatus is activated (Step 106), the enclosed apparatus generates the air-borne cleaning agent (exemplified by HOCl) from a solution (exemplified by sodium chloride solution). Activation can include (1) passing an electrolysis current between electrodes 207 in a sodium chloride solution in the container of the apparatus and (2) generating such agitation in the solution as to cause the release of droplets (microdroplets) of the solution and/or cleaning agent to become air-borne and exit from the opening in the lid of the container into the closed cabin to treat the air and surfaces of the cabin. The cleaning agent is allowed to dwell in the cabin for an effective time period to complete the treatment (Step 108). At that point the method is terminated, automatically by timer, or by remotely or manually stopping HOCl generation.

An exemplary embodiment of an apparatus useful in carrying out the systems and methods of the invention is illustrated in FIGS. 2A, 3A, 3B and 4A and 4B. As shown, the apparatus 200 has a container 210, with sides extending from a base 220 at one end and to an opening with a lid 250 at the other end. An opening 254 in the lid, such as a nozzle or vent, of the apparatus 200 is preferably adjustable in size and can be adjusted such that cleaning agent, represented by arrows 205 in FIG. 2B, accelerates upward through it, erupting from the opening with a trajectory depicted generally by arrows 205. The opening 254, as explained below, may have internal structures, like baffles 258, that remove entrained large liquid droplets from the air-borne stream of cleaning agent 205 as it exits from the apparatus 200. The exemplary container 210 has a diameter 212 at its upper end that is larger than its diameter 214 near its base 220. The base 220 contains a motor driven by either a battery pack (rechargeable or not) inside the base 220, or by electrical connection to an electrical outlet.

In this exemplary embodiment, microdroplets of the solution 201 (that will include HOCL generated by the electrolysis) is created by agitation of the solution. This agitation to cause air-borne microdroplets to be created may be provided by several means. For example, by use of an microdroplet generator 203 or an impeller 230. Non-limiting examples of the microdroplet generator 302 can include an atomizer or a pair or several pairs (multiple) of adjacent-spaced vibrating plates that cause localized strong agitation sufficient to create microdroplets and to cause solution 201 to become air-borne. Of course, other means may also be used as well as a combination of several means, such as an impeller 230 along with vibrating plates, and the like. In addition, in some cases the microdroplet generator 203 should have the capability to produce a gaseous cleaning agent.

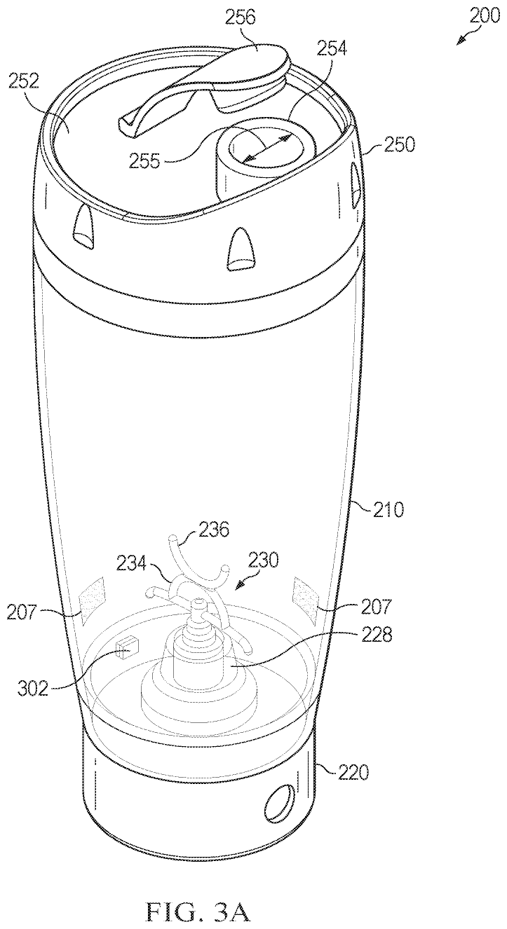

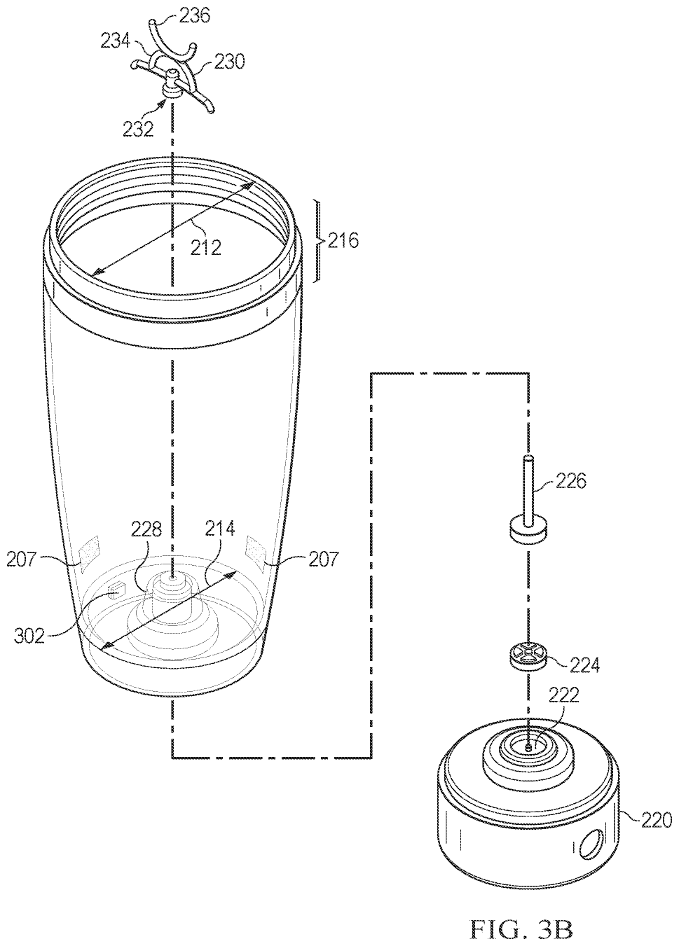

With reference to the impeller 230 shown in FIG. 3B, a spindle 226 is seated on an engaging wheel 224 that engages with motor spindle 222 and rotates in unison with motor spindle 222. An impeller 230 has a cavity 232 that friction fits to the spindle 226 so that the impeller 230 rotates as the spindle 226 rotates. The impeller 230 in the exemplary embodiment shown has a “double horse-shoe shape” with one horseshoe 234 curved downward, and the other horseshoe 236 curved upward so that the two are conjoined in a common plane at their respective apexes of curvature. This design facilitates creation of a vortex shape 272 when liquid in container 210 is agitated by the rotating impeller 230 in the container 210 at speed. A protective cover 228 shields the motor from contents of the container 210, and fits around the spindle 226, which projects out axially through a hole in the center of the cover 228. The spindle 226 is appropriately sealed against the hole to avoid or minimize leakage into a space under the cover 228.

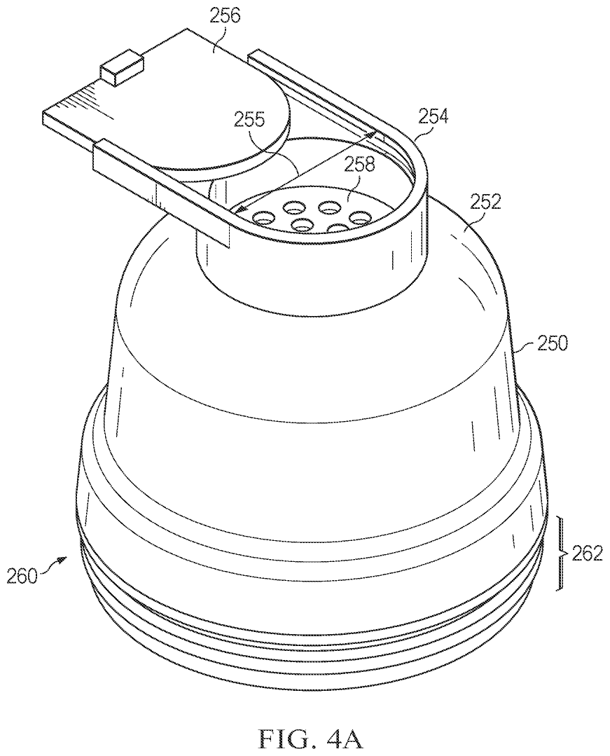

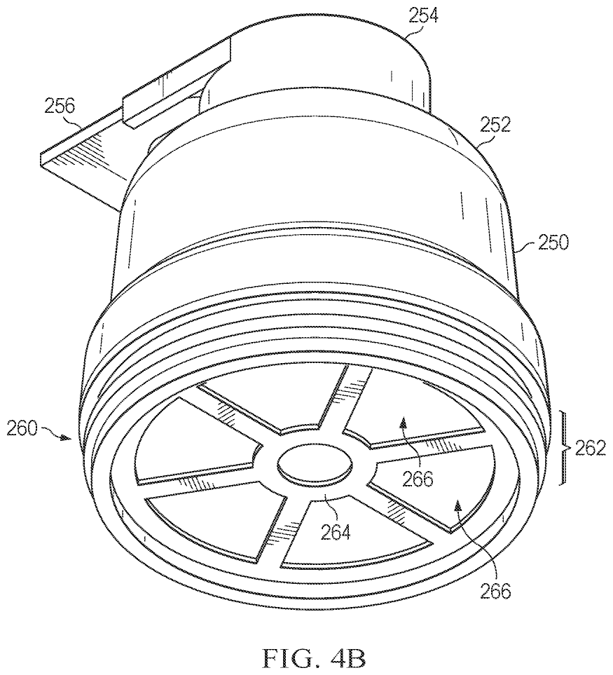

Referring more particularly to FIGS. 4A and 4B, an alternative illustrated exemplary embodiment of the lid 250 can be either friction fit to the upper end of the container 210 by engaging an upper lip of the container 210 or can be screwed onto the container 210 by threading 260 on the lower end 262 of lid 250 that engages corresponding threading on the container upper lip (not shown). The lid 250 has a top 252 that has an opening 254, equipped with a tab 256, extending from it that is preferably configured to adjust the cross-sectional area for flow through the opening.

Referring briefly back to FIG. 2A, when in use, the exemplary container 210 is partially filled with a solution 201, which includes chloride ions. In a non-limiting embodiment, the solution 201 can be a solution of sodium chloride. The cleaning agent 205 begins to evolve by electrolysis of the solution 201 when the apparatus 200 is activated and may accelerate due to agitation. Once sufficient cleaning agent 205 has evolved, the cleaning agent 205 erupts upward out through the nozzle as an air-borne spray to fill the enclosed space 180, which is vehicle cabin space in FIG. 2B, and commences attacking contaminant load on surfaces and malodor in the air.

Thus, the nozzle 254 has a variable sized inner diameter 255 shown in FIG. 4A that may be adjusted to cause air-borne cleaning agent emission from the container 200 through the nozzle 254 at a speed such that the emissions have both velocity and momentum to travel throughout the desired region of the cabin space to be cleaned. For example, the air-borne spray velocity is sufficient to travel through the passenger cabin of a car. In other embodiments, such as for a large SUV, a cabin of a tractor trailer rig, or hotel room either a larger apparatus or more than one apparatus may be needed to achieve total cabin permeation by the air-borne cleaning agent.

To avoid emitting foam and/or large droplets from the container, the lid 250 includes a baffle 264, exemplified in this non-limiting embodiment by a cart-wheel structure with spaces between the spokes covered with a fine mesh material 266, in its base area. In addition, the nozzle may include a further baffle 258, at its base, that includes perforations for flow of the cleaning agent 205.

While the cleaning agent 205 has been exemplified herein as hypochlorous acid (HOCl), other agents with similar properties may also be useful. For example, the properties of HOCl include that it is a disinfectant, but it is short-lived and denatures rapidly thus providing no long-term issues. HClO is classified as Non-Hazardous by the Environmental Protection Agency in the US. As with any oxidizing agent it can be corrosive or irritant depending on its concentration and pH. In a clinical test, hypochlorous acid water was tested for eye irritation, skin irritation, and toxicity. The test concluded that it was non-toxic and nonirritating to the eye and skin. While HOCl has oxidizing properties, alternatives, which may include reducing agents, with similar disinfection, safety and short-life properties, are also within the scope of this disclosure.

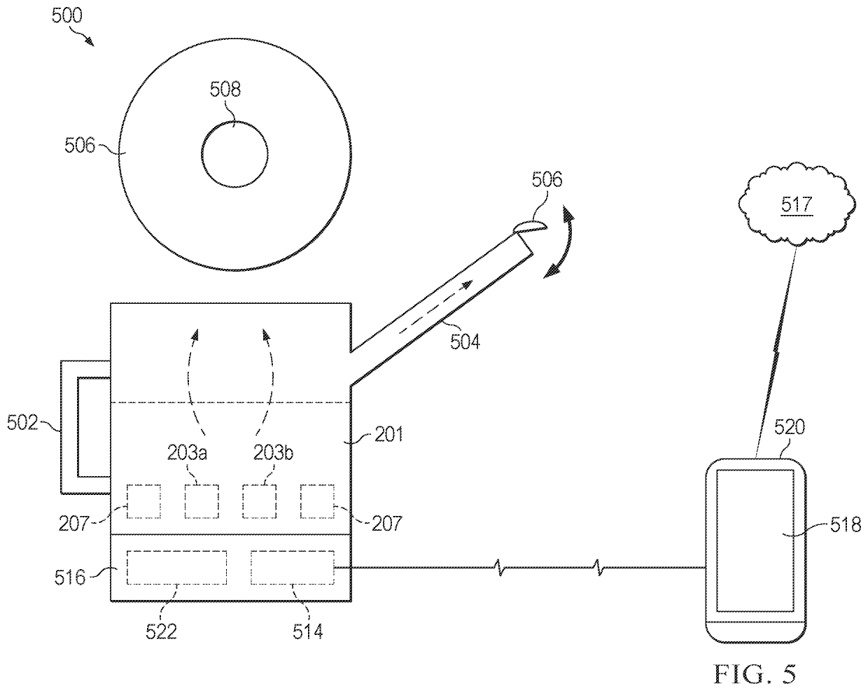

In exemplary embodiments the microdroplet generator 203 may comprise more than one microdroplet generator (one for larger microdroplets, the other for smaller) that may be selectively engaged or may include a microdroplet generator capable of adjustment to vary the microdroplet size. For example, in the non-limiting embodiment shown in FIG. 5, which is an apparatus 500 with a handle 502 for ease of use by an operator, if necessary, in “spray mode.” This mode may be used, for example, to clean drapes in a hotel room by emitting the air-borne cleaning agent in a directed manner via the nozzle 504 onto the drape surfaces. The air-borne cleaning agent can be created by larger droplet generator 203a.

The apparatus 500 is “dual mode” in that it can generate microdroplets of finer or of larger size, based on which generator is activated. In finer microdroplet mode, the nozzle 504 may be closed by a lid 506, and the vent hole 508 opened to a selected extent by using a variable sliding vent opening/closure mechanism. In finer microdroplet mode, the fine droplet generator 203b generates the air-borne cleaning agent, some of which may be in a gaseous state. Thus, the cleaning agent may be a mixture of microdroplets (comprising dissolved HOCl) and gaseous HOCl.

Electronic controls 514, which are shown housed in a base 516 of the apparatus 500, include means to connect to the internet 517 and/or to connect to an app 518 on a smart phone 520 of the operator. The app 518 may be used to activate the apparatus 500, shut it off, enter data about a particular cleaning project (e.g., VIN of car being cleaned, time and date, owner data, and cleaning protocol used, etc.) The apparatus 500 is powered by a battery 522, also housed in the base 516. Alternatively, the apparatus 500 can be powered by an electrical connection drawing power from a power supply in the enclosed space, such as an outlet.

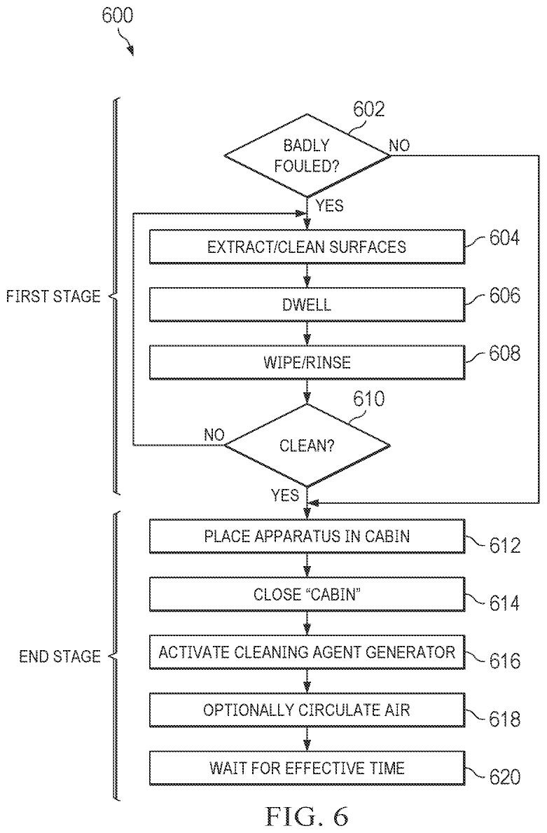

In some instances, a cabin is so fouled that use of the air-borne cleaning apparatus my be ineffective due to the high contaminant load. In these cases, an exemplary embodiment shown schematically in FIG. 6 of a multi-stage cleaning method 600 may be used. In an initial check, the extent of contaminant load is determined (Step 602). If the contaminant load is high, i.e., “badly fouled,” the surfaces of the cabin are treated with liquid cleaning agent that is configured to extract and remove contaminants from the surfaces and penetrates pores in the surfaces to extract contaminants (Step 604). The liquid cleaning agent may dwell on the surfaces to permit penetration into pores, dissolution of contaminants loosening of adhered debris, and other cleaning actions (Step 606). After a dwell period, the surfaces are rinsed to remove the cleaning agent and contaminant load that is has removed from the surfaces (Step 608). Inspection of the surfaces will determine whether the surfaces are “clean” to the desired degree (i.e., substantially free of contaminant load or not) (Step 610). If not, the process 600 is repeated until the surfaces are clean. Once the surfaces are clean, the end stage of the method 600 can be carried out to treat the cabin interior with the air-borne cleaning agent. The apparatus for generating an air-borne cleaning agent is placed in the cabin (Step 612). The cabin can be closed (Step 614). Closing the cabin can include closing the doors and windows of the cabin. The cleaning agent generator is activated (Step 616). Activation can include passing an electrolysis current between electrodes in a solution that can then yield a cleaning agent, such as HOCl, and can also include agitating the solution to. The air in the cabin can be optionally circulated throughout the cabin (Step 618). The cleaning agent is allowed to dwell in the cabin for an effective time period to complete the treatment (Step 620).

In addition, post cleaning of the cabin by HOCl, the cabin may be treated with odorizers to impart a pleasant smell to the interior of the cleaned cabin, or to mask any “chemical” smell.

### ADDITIONAL EMBODIMENTS

The following descriptive embodiments are offered in further support of the one or more aspects of the disclosure:

An exemplary embodiment is an apparatus for cleaning a contaminated enclosed space, the apparatus configured to generate a cleaning agent in situ in the enclosed space, the apparatus comprising:
a container configured for containing an electrolysis solution therein, when the apparatus is in use;
a lid configured to cover an opening of the container, the lid having a vent hole;
a pair of electrodes disposed in the container, the electrodes located for electrical communication when, during use, an electrolysis solution is contained in the container, such that a cleaning agent is created by electrolysis;
a generator configured to release a cleaning agent, the generator located in the container and in contact with the electrolysis solution when the apparatus is in use, the generator causing the cleaning agent to migrate into a space above a liquid level of the electrolysis solution and to be emitted from the vent hole of the lid into the enclosed space;
an electronic controller controlling the operations of the apparatus, including the generator and the electrodes; and
data transmission and receiving hardware associated with the electronic controller, the data receiving and transmission hardware transmitting data to and from the apparatus and a remote device comprising: a hand-held computing device with a user interface or, a smart phone, or a cloud computing system; and any one or more of the following limitations:
wherein when the apparatus is in use, the liquid comprises a solution of chloride ions at a concentration to generate hypochlorous acid when the pair of electrodes is activated;
a solution of chloride ions comprising chloride ions in a concentration that permits an evolution of HOCl at a rate sufficient for cleaning the cabin interior in 10 to 30 minutes;
wherein the electrolysis solution comprises sodium chloride;
wherein water used to make up the electrolysis comprises distilled water, deionized water, or water from reverse osmosis;
wherein the vent hole has an adjustable size to permit a buildup of pressure of the cleaning agent during use and to thereby permit controlled broadcasting of the cleaning agent in the enclosed space;
wherein the pair of electrodes are sized and spaced to permit an evolution of HOCl at a rate sufficient for cleaning the cabin interior in 10 to 30 minutes;
wherein the generator comprises a sonic generator of droplets;
wherein the generator comprises a piezoelectric droplet generator;
wherein the generator comprises an atomizer producing a mixture comprising a vapor of the cleaning agent and droplet comprising a mixture of the electrolysis solution and the cleaning agent;
wherein the generator comprises a capability to create droplets of an average size less than about 40 microns;
wherein the generator comprises a capability to create droplets of an average size greater than about 40 microns;
wherein the apparatus is activated by remote access of the electronic controller to activate the apparatus;
wherein the apparatus is deactivated by remote access of the electronic controller or by a programmable timer;
wherein the data transmitted comprises identifying data about the enclosed space being cleaned, and related data;
wherein the data transmitted comprises alerts;
a gas sparger located below a liquid level when an electrolysis solution is in the apparatus, the gas sparger configured to facilitate emitting a cleaning agent through the vent hole, when the apparatus is in use;
a motor-driven impeller, located below a liquid level when an electrolysis solution is in the apparatus, the impeller configured to agitate the electrolysis solution to facilitate emitting a cleaning agent through the vent hole, when the apparatus is in use;
wherein the container has an exterior configured to fit at least partially within a cup holder of a motor vehicle; and
wherein the cleaning agent generated is effective to eliminate or substantially reduce malodor caused by organic contamination of the enclosed space.

Another exemplary embodiment is a staged method of cleaning a contaminated enclosed space having contaminated surfaces therein, the method comprising:
identifying an enclosed space having surfaces therein that are contaminated;
applying to the contaminated surfaces a liquid first cleaning agent, the first cleaning agent configured to penetrate into pores, if any, of the contaminated surfaces to extract contaminants from the pores;
allowing the first cleaning agent to dwell on the contaminated surfaces and wet the surfaces for a first effective time;
wiping or rinsing off the first cleaning agent after the first effective time has elapsed to obtain cleaned surfaces;
identify whether the cleaned surfaces are substantially free of contaminants, and if not substantially free, repeat the steps of applying, allowing and wiping or rinsing;
placing an apparatus in the enclosed space;
closing off the enclosed space except that air recirculation may optionally be started through air ducts to be cleaned;
activating the apparatus to release a second air-borne cleaning agent into the enclosed space;
allowing the air-borne cleaning agent to dwell in the enclosed space for a second effective time; and
deactivating the apparatus after the second effective time has elapsed; and any one or more of the following limitations:
wherein in the step of covering the contaminated surfaces with a liquid first cleaning agent, the cleaning agent is an aqueous solution comprising a surfactant and a reducing agent;
wherein in the step of covering the contaminated surfaces with a liquid first cleaning agent, the cleaning agent is an aqueous solution comprising a surfactant and an oxidizing agent;
wherein the step of identifying an enclosed space having contaminants on surfaces comprises detecting a malodor caused by the contaminants;
wherein the step of identifying an enclosed space having contaminants on surfaces comprises detecting microbial contaminants;
wherein the step of activating the apparatus generates and expels an air-borne spray comprising chlorine dioxide;
wherein the step of activating the apparatus generates and expels hypochlorous acid into the enclosed space;
wherein the step of activating the apparatus comprises remote activation; and
wherein the apparatus is configured such that the second effective time is optionally set remotely, and the apparatus is deactivated automatically when the second effective time has elapsed.

While examples of embodiments of the technology have been presented and described in text and some examples also by way of illustration, it will be appreciated that various changes and modifications may be made in the described technology without departing from the scope of the inventions, which are set forth in and only limited by the scope of the appended patent claims, as properly interpreted, and construed.

What is claimed is:

1. A method of cleaning an enclosed space by in situ generation and distribution of hypochlorous acid into the enclosed space; the method comprising:
selecting an apparatus comprising a container;
placing the apparatus in the enclosed space;
activating generation of hypochlorous acid from the container of the apparatus;
distributing hypochlorous acid throughout the enclosed space as the hypochlorous acid is generated; and
allowing the distributed hypochlorous acid to dwell in the enclosed space for an effective time.

2. The method of claim 1, wherein the step of activating generation of hypochlorous acid comprises:
electrolyzing a solution comprising chloride ions in the container of the apparatus to release hypochlorous acid.

3. The method of claim 2, wherein the step of distributing the generated hypochlorous acid throughout the enclosed space comprises:
agitating the electrolyzing solution in the container to create fine air-borne liquid droplets and vapor comprising hypochlorous acid such as to emit the fine air-borne liquid droplets and vapor from the container and into the enclosed space.

4. The method of claim 3, wherein the step of agitating the electrolyzing solution in the container to create fine air-borne liquid droplets and vapor comprises bubbling a gas or gas mixture at a controlled rate through the electrolyzing solution at a rate that causes the generated hypochlorous acid to become air-borne as fine air-borne liquid droplets and vapor.

5. The method of claim 3, wherein the step of agitating the electrolyzing solution in the container to create fine air-borne liquid droplets and vapor comprises using ultrasonic vibration of the solution to create the fine air-borne liquid droplets and vapor.

6. The method of claim 3, wherein the step of agitating the electrolyzing solution in the container to create fine air-borne liquid droplets and vapor comprises using an atomizer taking suction from the solution in the container to create and disperse fine air-borne liquid droplets and vapor in the enclosed space.

7. The method of claim 1, wherein the enclosed space includes air ducts to be cleaned, and the method further comprises establishing fluid communication between the enclosed space and the air ducts such that hypochlorous acid circulates into the air ducts to clean the air ducts.

8. The method of claim 2 wherein the step of activating generation of hypochlorous acid comprises:
electrolyzing a solution comprising chloride ions in a concentration that permits an evolution of HOCl at a rate sufficient for cleaning the cabin interior in 10 to 30 minutes.

9. The method of claim 3, wherein the step of agitating to emit fine air-borne liquid droplets comprises droplets of an average size greater than about 40 microns.

10. The method of claim 3, wherein the step of agitating to emit fine air-borne liquid droplets comprises droplets of an average size less than about 40 microns.

11. The method of claim 1, wherein the step of allowing the distributed hypochlorous acid to dwell comprises allowing to dwell from about 5 to about 20 minutes.

12. The method of claim 1, wherein the cleaning comprises neutralizing or at least partially remediating malodor in the enclosed space.

13. The method of claim 1, wherein, after the step of allowing to dwell for an effective time, the apparatus is automatically powered down to stop the generation of hypochlorous acid.

14. The method of claim 1, wherein the apparatus is communicating with applications software on an electronic device or a cloud computing system remote from the apparatus.

15. The method of claim 14, wherein the applications software on the electronic device or the cloud computing system is accessible and controllable by an operator.

16. The method of claim 15, wherein the applications software on the electronic device or the cloud computing system is configured for storing stores identifying data about the enclosed space being cleaned, and storing date of cleaning, time of cleaning, and operator identifying data.

17. The method of claim 14, wherein the applications software on an electronic device or the cloud computing system is configured for providing alerts to an operator.

18. The method of claim 15, wherein the applications software on the electronic device or the cloud computing system is configured for allowing remote activating of generation of hypochlorous acid by an operator.

19. The method of claim 14, further comprising sensing of a concentration of hypochlorous acid in the enclosed space.

20. The method of claim 19, further comprising stopping the generation of hypochlorous acid when the effective time is reached and sending a notification via the applications software on an electronic device or the cloud computing system.
